(19) United States
(12) Patent Application Publication
(10) Pub. No.: US 2009/0197840 A1
(43) Pub. Date: Aug. 6, 2009

(54) COMPOSITION AND METHOD FOR ENHANCING SKIN CELL GROWTH, PROLIFERATION AND REPAIR

(75) Inventors: Alan E. KLIGERMAN, Egg Harbor Township, NJ (US); Margaret WEIS, Amarillo, TX (US)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: PRELIEF INC., Pleasantville, NJ (US)

(21) Appl. No.: 12/406,422

(22) Filed: Mar. 18, 2009

Related U.S. Application Data
(62) Division of application No. 11/934,305, filed on Nov. 2, 2007.
(60) Provisional application No. 60/864,040, filed on Nov. 2, 2006.

(51) Int. Cl. A61K 31/66

(52) U.S. Cl. 514/129

(57) ABSTRACT

A skin treatment composition is provided which contains calcium glycerophosphate and a fatty acid source derived from an animal or a vegetable. The composition enhances skin cell proliferation and growth and repair of damage to skin cells. Methods for enhancing skin cell repair, proliferation, and growth and for enhancing ceramide synthesis involve topically applying to the skin a composition containing calcium glycerophosphate and a fatty acid source derived from an animal or a vegetable.

### CROSS-REFERENCE TO RELATED APPLICATIONS

This application is a divisional of U.S. patent application Ser. No. 11/934,305, filed Nov. 2, 2007, now abandoned, which claims the benefit of U.S. Provisional Patent Application No. 60/864,040, filed Nov. 2, 2006, the disclosures of which are herein incorporated by reference in their entirety.

### BACKGROUND OF THE INVENTION

The key roles of certain fatty acids (FA), primarily C16:0 (palmitic acid) and C4:0 (butyric acid), in the regulation of skin cells are well known. These acids are essential in the synthesis of key cellular components, such as ceramide, a sphingolipid which is a vitally important signaling molecule as well as a structural component of cell membranes. Vegetable source fats have long been applied to skin in association with many other components. In contrast, animal source fats are seldom applied to the skin for a number of reasons, including difficulty in fractionating off their specific valuable acids, e.g., butyric and palmitic, difficulty in handling isolated butyric acid, and the heretofore problematic odors associated with such animal source fatty acids.

The central fatty acid for sphingolipid synthesis is palmitic acid, via its derivative palmitoyl-CoA. The first reaction in sphingolipid synthesis is the condensation of palmitoyl-CoA with the amino acid serine, followed by a stepwise transformation through dehydrosphingosine, dihydrosphingosine and finally sphingosine to ceramide. The second position of sphingosine may be filled by any fatty acid: short chain (e.g. butyric, caproic, octanoic, etc.), medium chain (up to 12 carbons), or long chain (14 carbons and above).

Present knowledge suggests that the nature of the fatty acid at the second position of sphingosine determines whether the specific ceramide thereby formed becomes structural or signaling. The literature suggests that introduction of exogenous short chain fatty acids stimulates the production of signaling ceramides, but does not offer insight as to whether those short chain fatty acids are incorporated into the signaling ceramides' own structure as well (J. Lipid Res., 46(9):1944-1952, 2005).

Many other fatty acids are involved in the synthesis and function of glycerophospholipids, which include a number of entities having both structural and signaling functions. Structurally, phospholipids are the fundamental building blocks of the cell membrane. With respect to signaling, lipids, such as diacylglycerol, and lipid derivatives, such as prostaglandins, are important regulators of multiple functions.

Glycerophospholipids nearly always contain a saturated fatty acid at their first position and an unsaturated or polyunsaturated fatty acid at their second position. Obviously, then, both types of fatty acids are important. It is clear that the fatty acids are not randomly assigned to glycerophospholipids or sphingolipids and that the importance of fatty acids is, counter-intuitively, not a function of the amount or concentration thereof. For example, even though arachidonic acid is a relatively small fraction of the total fatty acid in glycerophospholipids and sphingolipids, it is immensely important because it serves as the precursor for prostaglandins. Prostaglandins are another important group of signaling molecules that mediate a wide range of physiological functions, such as control of blood pressure, contraction of smooth muscle, and modulation of inflammation.

How fatty acids are assigned to a particular location is a subject of intense debate, and is one of the current laboratory foci of some workers in the field. At present, there is sufficient evidence to indicate that specificity in fatty acid utilization may be influenced by the local metabolic milieu. Thus, it is possible that fatty acids (or other molecules), even those that are not directly incorporated into glycerophospholipid or sphingolipid entities, might nevertheless influence the fatty acid composition of those lipids, depending upon the operating environment (Psychopharmacology 184: 122-9, 2006).

The roles of certain fatty acids, especially C4:0 (butyric acid) and C16:0 (palmitic acid) in skin cells are highly specific: they promote keratin production via signaling through sphingosine-1-phosphate (S1P) to synthesize ceramide. Ceramide is a molecule of transcendent importance which participates in an essential signal delivery mechanism that, among other functions, signals keratinocytes to undergo apoptosis to corneal skin cells. S1P, however, is readily dephosphorylated into sphingosine, unless there is some agent present to block or prevent that dephosphorylation. In itself, sphingosine has no known signaling function.

### BRIEF SUMMARY OF THE INVENTION

skin treatment composition is provided which comprises calcium glycerophosphate and a fatty acid source derived from an animal or a vegetable. A method for enhancing skin cell repair, proliferation and growth comprises topically applying to the skin a composition comprising calcium glycerophosphate and a fatty acid source derived from an animal or a vegetable.

Methods of enhancing insertion of animal or vegetable source fatty acids (short chain, medium chain, and/or long chain) into a cellular process, of enhancing ceramide synthesis, and of enhancing reproduction, differentiation, proliferation, growth, repair, programmed apoptosis and health of skin cells comprise topically applying to the skin a composition comprising calcium glycerophosphate and a fatty acid source derived from an animal or a vegetable.

### BRIEF DESCRIPTION OF THE SEVERAL VIEWS OF THE DRAWINGS

The foregoing summary, as well as the following detailed description of the invention, will be better understood when read in conjunction with the appended drawing. For the purpose of illustrating the invention, there is shown in the drawing an embodiment which is presently preferred. It should be understood, however, that the invention is not limited to the precise arrangements and instrumentalities shown.

In the drawings:

FIG. 1 is a schematic diagram of the enhanced synthesis of ceramide utilizing calcium glycerophosphate and a short chain fatty acid.

### DETAILED DESCRIPTION OF THE INVENTION

The present invention is directed to a composition for topical application to the skin which provides for enhanced skin cell proliferation, growth, differentiation, and repair. The composition comprises calcium glycerophosphate and a fatty acid source derived from an animal or a vegetable.

Calcium glycerophosphate (CGP) is also known as 1,2,3-propanetriol, mono(dihydrogen phosphate) calcium salt (1:1), calcium glycerinophosphate, calcium phosphoglycerate and Neurosin. It may exist as a hydrate, including the monohydrate and the dihydrate. Three CGP isomers exist, namely β-glycerophosphoric acid calcium salt ((HOCH₂)₂CHOPO₃Ca) and D(+) and L(−)-α-glycerophosphoric acid calcium salt (HOCH₂CH(OH)CH₂OPO₃Ca). Any one isomer, or any combination of two or more isomers, may be used as the CGP according to this invention. A commercially available form of CGP is a mixture of calcium β- and DL-α-glycerophosphates, and this is a preferred form of CGP according to the invention. The preferred form of CGP is food grade CGP according to Foods Chemical Codex (FCC) III, and may be obtained from Astha Chemical Co., Hyderabad, India; Seppic Inc., Fairfield, N.J., as well as Gallard Schlesinger Company, Carl Place, N.Y. 11514, which is a distributor for the Dr. Paul Lohmann GmbH KG of Emmerthal, Germany.

The fatty acid source for use in the composition may be derived from an animal, preferably from butter, butter serum, or butter oil. In small quantities, butter may be obtained at any supermarket, and larger quantities are available from any dairy wholesaler. Non-refrigerated “Golden Churn” brand and “Red Feather” brand (FDA-approved) shelf-stable butter, each of which contains 81% butterfat, may be obtained from Ballantyne Goods Pty Ltd. (39 Ballantyne Street, South Melbourne, VIC 3205 Australia), and butter serum may be obtained from Solarec S.A. (Rte. de Saint Hubert 75, Recogne, Libramont-Chevigny B-6800, 1011077121, Belgium). While cow's milk (bovine) butter is preferred, it is also within the scope of the invention to utilize butter made from the milk of any other mammals, including sheep, yak, goat, and conceivably, even human. Each species contains its own unique fatty acid ratio. Other animal fats, such as suet and lard, contain virtually no short-chain fatty acids, and thus would not be preferred for use in the inventive composition.

Although plant-derived materials, including plant fats, have long been utilized in cosmetics, there are some cases in which animal fats are superior to vegetable fats because the former are rich sources of short chain fatty acids, such as butyric acid. Furthermore, while plant sterols are abundant, only animal fats contain cholesterol, the specific sterol that is an essential component of the animal cell membrane. Accordingly, for some applications, the composition according to the invention may desirably contain a fatty acid source derived from an animal. The term “fatty acid source derived from an animal” may be understood to also encompass fatty acids which mimic those derived from an animal. For example, very short chain fatty acids, including butyric acid, can be derived from bacterial fermentations. For example, propionic acid is produced by Propionobacter sp. fermentation of milk solids, as in the production of Swiss Cheese. Preferably, the fatty acid source contains short, middle, and long chain fatty acids.

It is also within the scope of the invention to include in the composition a fatty acid derived from a vegetable source. A wide variety of vegetable sources of fatty acids are known and include, without limitation, oils and/or butters (or similar derivatives) derived from almond, apricot kernel, argan, avocado, babassu, black current seed, borage, camellia seed, canola, carrot, castor, cherry kernel, cocoa, coconut, cotton seed, evening primrose, flax seed, grape seed, hazelnut, hemp seed, jojoba, ku kui nuts, linseed, macadamia nuts, meadowfoam, neem, palm, olive, passion fruit, pomace, palm kernel, peach kernel, pecan, perilla seed, pomegranate, poppy seed, pumpkin seed, rice bran, rose hip, safflower, sea buckthorn, sesame, shea, soya bean, sunflower, tamanu, walnut, and wheat germ.

Vegetable oils, butters, and other derivatives may be commercially obtained from, among other sources: Herbal Accents, P.O. Box 937, Alpine, Calif. 91903-0937; Mountain Rose Herbs, P.O. Box 50220, Eugene, Oreg. 97405 and dozens of other small and large suppliers.

It is further within the scope of the invention to utilize a synthetic mixture of fatty acids which would mimic those derived from an animal or a vegetable. Such a synthetic mixture would also be encompassed by the phrase “fatty acid source derived from an animal or a vegetable.” Such mixtures would have the advantage of having exact and unvarying fatty acid compositions and fixed cholesterol concentrations. However, these synthetic mixtures may be very expensive and thus economically restrictive. Alternatively, a mixture of animal and synthetic fats may be utilized to optimize cost and quality target criteria. For example, a synthetic fatty acid/cholesterol/fatty ester/cholesterol ester mix may be desirable.

The fatty acid source, whether of animal or vegetable origin, is preferably present in the composition at a concentration of about 0.1 to 75% by weight, more preferably about 10% by weight of the composition. The calcium glycerophosphate is preferably present at a concentration of about 0.1 to about 50% by weight, more preferably about 6.75 to 7.5% by weight of the composition. In addition to the CGP and fatty acid source, the composition contains a suitable cosmetic carrier or vehicle, preferably a water-based carrier for providing a “wet” composition or a carrier such as cornstarch for providing a “dry” composition.

A variety of additional components and additives may be included in the composition, and these may include mixtures of animal and vegetable fatty acid sources. For example, it may be desirable to include a moisturizer, such as glycerin, olive oil, isopropyl stearate, isopropyl palmitate, isopropyl myristate, sorbitol, lanolin, etc. Stabilizers or gel formers may be included, such as cellulose gum, any hydrocolloid, alginate or marine source colloid, or more generally any food grade or cosmetic grade rheologically-governing product known in the art. It may be desirable to include a preservative, such as methyl paraben, a propionate, nitrate, nitrite, benzoate, sorbate, or other paraben (an ester of p-hydroxybenzoic acid).

It is preferred to include at least one anti-oxidant in the composition to prevent degradation of the fatty acids and the development of undesirable odors during product storage. Appropriate antioxidants include synthetic antioxidants, such as butylated hydroxyanisole (BHA), butylated hydroxytoluene (BHT), propyl gallate (PG), and tert-butylhydroquinone (TBHQ), and natural antioxidants, such as flavonoids, polyphenols, ascorbic acid (Vitamin C) or tocopherols (Vitamin E).

Additional body-modifying, smoothing, moisture-barrier and skin-forming components may be included, such as silicone or silicone-content products that are commercially formulated and available to food and cosmetic manufacturers. For example, cosmetic grades of silicones such as Dow Corning Silky Touch® product ST-Cyclomethicone 5-NF (decamethylcyclopentasiloxane), which is one of fourteen silicones commercially available from Dow Corning for topical application (Dow Corning Corporation, 2200 W. Salzburg Road, PO Box 994, Midland, Mich. 48686-0994), may be used. Also, Gransil DM5® is a polydimethylsiloxane and cross-linked silicone polymer (commercially available from Grant Industries, Inc.; 103 Main Avenue, Elmwood Park, N.J. 07407).

It may also be desirable to include fragrances and/or coloring agents in the composition. Additives of such types which are appropriate for cosmetic products are well-known in the art and need not be described.

It is also within the scope of the invention to include an antibacterial/antibiotic agent in the composition, which makes the composition particularly appropriate for application to skin surfaces containing open wounds or other compromised skin conditions. For example, iodine or iodine combinations (povidone, Betadyne®, etc.) and other widely-used, non-prescription products such as bacitracin, neomycin, mupirocin, and polymyxin B may be included. Commercially available products which contain these ingredients include Neosporin®, Polysporin®, and Triple Antibiotic® Ointment or Cream. Also utilizable as a prescription antibiotic is Bactroban®.

Finally, it may be desirable to include an anesthetic, such as amethocaine, lidocaine, or prilocalne, for treating pain. However, it has been found that on some wounds, particularly superficial burns, the use of one of the primary active ingredients, CGP, acts as a pain relief agent in and of itself.

It should be noted that the specific compounds which have been described herein as examples of the optional components, including moisturizers, stabilizers, preservatives, antioxidants, body-modifiers, moisture barriers, fragrances, coloring agents, antibacterial/antibiotic agents, and anesthetics, are meant to be representative and exemplary of compounds which fall into each of these classes. These compounds are not intended to be limiting, and the use of additional or alternative additives and compounds in these classes which are known in the art or to be developed and which are appropriate for skin compositions is also within the scope of the invention.

The pH of the composition is preferably above about 4, more preferably about 4.5 to about 7, even more preferably about 5.55 to about 5.8, and most preferably about 5.65. The pH may be adjusted by including a suitable buffer or buffering agent, such as lactic acid, preferably D, L-lactic acid. Dry lactic acid powder is commercially available from Musashino Chemical Laboratory, Ltd. (Kyobashi, Chuo-Ku, Tokyo, Japan) and Penta Manufacturing Company (Livingston, N.J. 07039); while this lactic acid is not a D, L racemate, it will still function as an appropriate pH adjuster. If ascorbic acid (vitamin C) is included in the composition as an antioxidant, the level of lactic acid in the formulation must be appropriately reduced to achieve the desired pH level.

The composition may be prepared in a variety of forms, including without limitation a cream, ointment, salve, unguent, paste, lotion, and dry powder. It may also be incorporated into a wet “wipe” or moistened towelette. In some situations, such as when there is damaged skin that is already moist from any substance, including blood, the dry powder may be directly applied to the skin. In other cases, a dry powder may be reconstituted with water and then applied to the skin, or added to a soak or bath water.

It is also within the scope of the invention to administer the composition in conjunction with a known therapeutic, pharmaceutical, or cosmetic product, such as a powder, spray, ointment, cream, gel or lotion. For instance, the composition may be used as a vehicle for a prescription or over-the-counter pharmaceutical topical preparation, such as a psoriasis remedy, a poison ivy/sumac product, a dermatitis product, an insect bite product, a burn treatment product, a sunburn treatment product, or a product intended to ameliorate topical skin damage from radiation-type therapies. It may also be used as a vehicle for a topical bactericide, such as alcohol, povidone, Bacitracin®, or Neosporin®, a fungicide, such as an athlete's foot product, an anti-itch product, an anti-rash product, an anti-chafing product, or an anti-perspirant. It may also be desirable to utilize the composition as a vehicle for or in conjunction with a cosmetic, such as a moisturizer, sun block, sun damage product, “wrinkle remover”, anti-aging product, anti-stretch mark product, lipstick, lip balm, eye-shade, makeup product, makeup remover, cosmetic foundation, deodorant, pre- and after-shave product, artificial blush, mascara, or artificial suntan product, a hair product, such as a shampoo, hair treatment, conditioner, grooming product, or hair dye, or a personal care product, such as a soap, shaving cream, feminine hygiene product, lubricant product, etc.

The composition may also be used as an immediate anti-irritant after certain body treatments, such as a mud bath, bath salt, bubble bath, body massage astringent, chemical or mechanical skin peel, for example. The composition may also be utilized to minimize sensitive external and/or internal epithelial skin irritation, including but not limited to those occasioned by sexual contact, with application to one partner having a beneficial effect on the other partner via its incidental contact, amounting to secondary topical application.

The composition may be applied to the skin as frequently as desired: once, twice, three times a day or more, depending on the user's desire. There is no possibility of an “overdose.” The composition may be applied to whichever areas of the body require treatment, including hair, head, face, lips, nose, ears, neck, shoulders, underarm, trunk, pelvic regions including genital, vaginal and peri-anal areas, legs, feet, or between toes, for example. Thus, for the purposes of this disclosure, the phrase “skin treatment composition” may be understood to refer to a composition which may be applied to any external area of the body, even if the area is not skin per se (i.e., hair, lips, etc.). It may be applied to an area of the body covering less than one square inch (less than ˜6.5 cm²) or to a much larger body area, including entire limbs or the entire back, for example. It is also within the scope of the invention to incorporate the composition into a household or other utilitarian product which is not intended for personal use but which contacts the skin directly, such as a dish detergent, cleanser, general all-purpose cleaner, cleaning wipe, etc.

The invention also relates to a method for enhancing skin cell proliferation, growth, and repair which comprises topically administering to the skin a composition comprising calcium glycerophosphate and a source of fatty acids derived from an animal or a vegetable, as previously described.

A variety of additional methods are also included in the invention. For example, a method of enhancing ceramide synthesis comprises topically applying to the skin a composition comprising calcium glycerophosphate and a fatty acid source derived from an animal or vegetable as previously described. This composition enhances ceramide synthesis by enhancing movement of sphingomyelin to ceramide, by enhancing conversion of phosphatidyl choline to phosphocholine and of phosphocholine to sphingomyelin, and by inhibiting dephosphorylation of spingosine-1-phosphate. A method of enhancing insertion of animal or vegetable source fatty acids (short chain, medium chain, and/or long chain) into a cellular process comprises topically applying to the skin a composition comprising calcium glycerophosphate and a fatty acid source derived from an animal or vegetable as previously described.

Further, a method of enhancing reproduction, differentiation, proliferation, growth, repair, programmed apoptosis and health of skin cells comprises topically applying to the skin a composition comprising calcium glycerophosphate and a fatty acid source derived from an animal or vegetable as previously described. The composition enhances signaling in the reproduction, differentiation, proliferation, growth, repair, programmed apoptosis and health of the skin cells and also contributes structural substances to the reproduction, differentiation, proliferation, growth, repair, programmed apoptosis and health of the skin cells.

Applicants have discovered the unexpected results which are achieved by combining calcium glycerophosphate and a source of animal fatty acids, such as butter or its derivatives, which contains short, medium, and long chain fatty acids. Superior results are also achieved when combining calcium glycerophosphate with vegetable oils or vegetable oil combinations of the same characteristics. Application of such compositions is attractive because it provides a range of utilizable extrinsic fatty acids to skin cells. Simultaneously, the CGP-contributed components of the composition provide both up-regulation of desirable cellular growth-proliferation reproduction signaling and down-regulation of anti-proliferative-reproductive signaling. The composition thus provides unique benefits to the reproduction, differentiation, proliferation, growth, repair, programmed apoptosis and health of cells.

Without wishing to be bound by theory, it is believed that this combination will be effective because glycerophosphate (GP) prevents or inhibits dephosphorylation of SIP and thus amplifies the SIP effect, which is the signal-accelerated synthesis of ceramide from sphingosine into ceramide, and is also an inhibitor of the down-regulating serine/threonine phosphatases in keratin differentiation and reproduction. Calcium is one of the signals in the biochemical pathway that activates ceramide synthesis. Therefore, since calcium glycerophosphate supplies both calcium and glycerophosphate, it provides a double amplification by enhancing both the S1P and keratin phosphorylation activities. The amount of calcium supplied by a topically-applied CGP-content skin vehicle lotion is more than adequate, given that the level of calcium required for cellular signaling is in the nanomolar range. The particular combination of calcium and glycerophosphate in these roles is the subject of U.S. patent application Ser. No. 10/639,213 of Applicants, the disclosure of which is incorporated herein by reference.

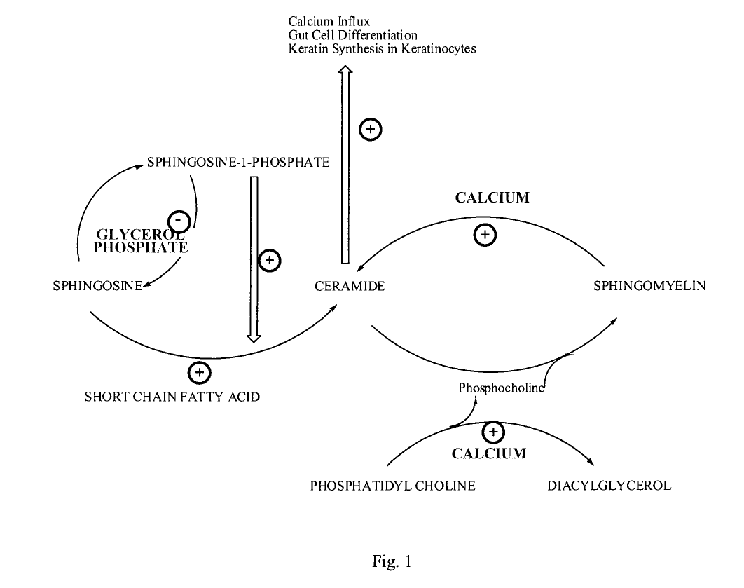

Following ceramide synthesis, keratinocytes undergo apoptosis as they terminally differentiate and move into the stratum corneum. Protein kinase-C phosphorylates proteins, an essential process for such differentiation. The sphingolipid ceramide activates the atypical protein kinase-C isoform, and the free ion calcium initiates steps required for ceramide synthesis, and both are therefore essential for apoptosis. As described in the '213 application, GP is an inhibitor of protein phosphatases in keratinocyte differentiation, and so the protein kinase-C effect would be magnified in that proteins would be more likely to phosphorylate and would be less likely to be dephosphorylated. A schematic diagram of the enhanced synthesis of ceramide utilizing calcium glycerophosphate and a short chain fatty acid, such as butyric acid, is depicted in FIG. 1.

As shown in FIG. 1, both sphingosine and sphingomyelin move toward ceramide synthesis. Sphingosine is partially phosphorylated into SIP, which signals (downward arrow) an acceleration (+) of the synthesis of ceramide from sphingosine. Some SIP is dephosphorylated back to sphingosine, an event inhibited (−) by GP. Short chain fatty acids are further signalers (+) and/or are incorporated into ceramide. Ceramide then signals (+) for various vital cell functions (upward arrow). Transfer of phosphocholine from phosphatidyl choline to ceramide results in sphingomyelin synthesis, a process which is accelerated by calcium (+). In turn, sphingomyelin is converted back to ceramide by the action of the calcium activated enzyme, sphingomyelinase.

In addition to contributing to the health of normal skin, it is believed that the combination of fatty acids and calcium glycerophosphate is valuable in repairing damaged skin by contributing to the defense mechanism of blood platelets. Platelets are one of the formed elements of the blood whose function is to stop bleeding and to release factors that promote wound healing. When tissue is damaged, platelets become activated and stick together to form a mechanical barrier which stops blood loss. At the same time, they release a number of factors that strengthen the mechanical barrier and recruit the various cell types required to repair the damage. S1P is produced by platelets and released when platelets are activated. S1P promotes the migration of new vascular endothelial cells (the cells that line the blood vessels), stimulates the proliferation of fibroblasts (the cells responsible for the formation of scar tissue) and facilitates the re-formation of the epithelial barrier. All of these events take place in the blood vessels. As noted above, it is believed that glycerophosphate prevents the dephosphorylation of S1P and thus amplifies the S1P effect. As shown in FIG. 1, both CGP and short chain fatty acids enhance ceramide synthesis, consequently enhancing keratinocyte differentiation (J. Biol. Chem. 279:38471, 2004; Br. J. Dermatol. 151:961, 2004; J. Clin. Invest. 108:689, 2001).

In conclusion, the combination of CGP and animal and/or vegetable source fatty acids is unique and favorable for topical application to the skin. The CGP delivers proliferation and growth signals and simultaneously down-regulates certain cell signaling inhibitors, while also contributing molecular substance; the fatty acids deliver important proliferation and growth signals while also contributing molecular substance. The combination of the two, topically applied, is a unique formulation for reproduction, differentiation, proliferation, growth, repair, programmed apoptosis and health of skin cells.

The invention will now be described by the following, non-limiting examples. Although these examples do not include antibiotics, anesthetics, antioxidants, fragrances, coloring agents, or body-modifiers, it is also within the scope of the invention to include such components in the compositions described below.

### Example 1

### Preparation of a Composition Containing 8% Olive Oil and 7.5% CGP

| Components | | Role |
|---|---|---|
| 8% | olive oil* | fatty acid source |
| 7.5% | CGP | cellular repair, growth, and |
| | | proliferation stimulator |
| 1.8% | DL-lactic acid | buffer |
| 2.5% | cellulose gum | stabilizer |
| 1.0% | glycerin | moisturizer |
| 0.2% | methylparaben | preservative |
| 79% | (balance) water | carrier |

*All percentages are by weight

Water and glycerin are combined in a 500 ml glass beaker, then combined with a mixture of the dry ingredients (CGP, methyl paraben, and cellulose gum). The resulting mixture is mixed using a high speed shear mixer (Braun Multiquick MR 300) for 2 minutes at room temperature. The pH is then adjusted with the lactic acid to a pH of 5.75. The olive oil is added and blended using a hand instrument to produce a product having an ointment-like consistency.

### Example 2

### Preparation of a Composition Containing 50% Fatty Acid Source and 3.75% CGP

| Components | Role |
|---|---|
| 50 grams butter, 50 grams olive oil, or | fatty acid source |
| 25 grams olive oil and 25 grams coconut oil | |
| 3.75 grams calcium glycerophosphate | cellular repair, growth, and |
| | proliferation stimulator |
| 0.5 grams glycerin | moisturizer |
| 1.38 grams cellulose gum | stabilizer |
| 0.1 grams methyl paraben | preservative |
| 0.95 grams lactic acid solution | buffer |
| (88% by weight) | |
| Balance water (to yield 100 g product) | carrier |

Water and glycerin are combined in a 500 ml glass beaker, then combined with a mixture of the dry ingredients (CGP, methyl paraben, and cellulose gum). The resulting mixture is mixed using a high speed shear mixer (Braun Multiquick MR 300) for 2 minutes at room temperature. The pH is then adjusted with the lactic acid solution to a pH of 5.65. The butter or oil(s) are added and blended using a hand instrument to produce a product having an ointment-like consistency.

### Example 3

### Preparation of a Composition Containing 25% Fatty Acid Source and 5.625% CGP

| Components | Role |
|---|---|
| 25 grams butter or 25 grams canola oil | fatty acid source |
| 5.625 grams calcium glycerophosphate | cellular repair, growth, |
| | and proliferation stimulator |
| 0.75 grams glycerin | moisturizer |
| 2.07 grams cellulose gum | stabilizer |
| 0.15 grams methyl paraben | preservative |
| 1.425 grams lactic acid solution | buffer |
| (88% by weight) | |
| Balance water (to yield 100 g product) | carrier |

Water and glycerin are combined in a 500 ml glass beaker, then combined with a mixture of the dry ingredients (CGP, methyl paraben, and cellulose gum). The resulting mixture is mixed using a high speed shear mixer (Braun Multiquick MR 300) for 2 minutes at room temperature. The pH is then adjusted with the lactic acid solution to a pH of 5.65. The butter or canola oil is added and blended using a hand instrument to produce a product having a thin ointment or thick lotion-like consistency.

### Example 4

### Preparation of a Composition Containing 10% Fatty Acid Source and 6.75% CGP

| Components | Role |
|---|---|
| 10 grams butter or 10 grams shea butter oil | fatty acid source |
| 6.75 grams calcium glycerophosphate | cellular repair, growth, and |
| | proliferation stimulator |
| 0.9 grams glycerin | moisturizer |
| 2.49 grams cellulose gum | stabilizer |
| 0.18 grams methyl paraben | preservative |
| 1.71 grams lactic acid solution | buffer |
| (88% by weight) | |
| Balance water (to yield 100 g product) | carrier |

Water and glycerin are combined in a 500 ml glass beaker, then combined with a mixture of the dry ingredients (CGP, methyl paraben, and cellulose gum). The resulting mixture is mixed using a high speed shear mixer (Braun Multiquick MR 300) for 2 minutes at room temperature. The pH is then adjusted with the lactic acid solution to a pH of 5.65. The butter or shea butter oil is added and blended using a hand instrument to produce a product having a lotion-like consistency.

### Example 5

### Preparation of a Composition Containing 10% Fatty Acid Source and 7.5% CGP

| Components | Role |
|---|---|
| 10 grams dry butter powder or 10 | fatty acid source |
| grams room temperature coconut oil | |
| 0.50 grams granulated CGP | cellular repair/growth and |
| (mesh size < 100) | proliferation stimulator |
| 2.6 grams glycerin powder | moisturizer |
| 4.98 grams cellulose gum | stabilizer |
| 0.36 grams methyl paraben | preservative |
| 0.94 grams lactic acid | buffer |
| Balance dry carrier (such as corn starch) | carrier |
| to yield 100 g product | |

All of the ingredients are mixed dry using a Kitchen-Aid mixer at slow speed with wire whisk beaters. The resulting product is a dry powder which is suitable for wetting at the site of application or for direct application to an already wetted skin surface.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method for enhancing skin cell repair, proliferation, and growth comprising topically applying to the skin a composition comprising calcium glycerophosphate and a fatty acid source derived from an animal or a vegetable.

2. The method according to claim 1, wherein the skin is moist and the composition is applied as a dry powder.

3. The method according to claim 1, wherein the composition is in a form of a dry powder, further comprising combining the composition with water or adding the composition to bath water prior to applying the composition to the skin.

4. The method according to claim 1, wherein the fatty acid source comprises short, medium, and long chain fatty acids.

5. The method according to claim 1, wherein the fatty acid source comprises cholesterol.

6. The method according to claim 1, wherein the fatty acid source is derived from butter or a derivative thereof.

7. The method according to claim 1, wherein the composition is in a form selected from the group consisting of a cream, ointment, salve, unguent, paste and lotion.

8. The method according to claim 1, wherein the composition further comprises at least one component selected from the group consisting of a fragrance, a coloring agent, a body modifying component, an antibacterial/antibiotic agent, an anesthetic, an antioxidant, a stabilizer, a moisturizer, a preservative, and a buffering agent.

9. The method according to claim 1, wherein the composition has a pH of about 4.5 to 7.

10. The method according to claim 1, wherein the composition further comprises a synthetic acid source.

11. The method according to claim 1, wherein the composition is administered in conjunction with a known product selected from the group consisting of a cosmetic product, a pharmaceutical product, and a therapeutical product.

12. A method of enhancing ceramide synthesis, the method comprising topically applying to the skin a composition comprising calcium glycerophosphate and a fatty acid source derived from an animal or a vegetable.

13. The method according to claim 12, wherein the composition inhibits dephosphorylation of spingosine-1-phosphate.

14. The method according to claim 12, wherein the composition enhances movement of sphingomyelin to ceramide.

15. The method according to claim 12, wherein the composition enhances conversion of phosphatidyl choline to phosphocholine and of phosphocholine to sphingomyelin.

16. The method according to claim 12, wherein the skin is moist and the composition is applied as a dry powder.

17. The method according to claim 12, wherein the fatty acid source comprises short, medium, and long chain fatty acids.

18. The method according to claim 12, wherein the fatty acid source comprises cholesterol.

19. The method according to claim 12, wherein the fatty acid source is derived from butter or a derivative thereof.

20. The method according to claim 12, wherein the composition is in a form of a dry powder, further comprising combining the composition with water or adding the composition to bath water prior to applying the composition to the skin.
